(19) United States
(12) Patent Application Publication
(10) Pub. No.: US 2002/0173511 A1
(43) Pub. Date: Nov. 21, 2002

(54) Serotonergic compositions and methods for treatment of mild cognitive impairment

(76) Inventors: Richard J. Wurtman, Boston, MA (US); Robert K. K. Lee, Boston, MA (US)

Correspondence Address: Patent Administrator, KATTEN MUCHIN ZAVIS, Suite 1600, 525 West Monroe Street, Chicago, IL, 60661-3693, US

(21) Appl. No.: 09/986,469

(22) Filed: Nov. 8, 2001

Related U.S. Application Data
(60) Provisional application No. 60/246,615, filed on Nov. 8, 2000.

(51) Int. Cl. A61K 31/496; A61K 31/495; A61K 31/454; A61K 31/46; A61K 31/4535; A61K 31/353; A61K 31/405; A61K 31/155; A61K 31/135

(52) U.S. Cl. 514/252.12; 514/254.02; 514/304; 514/255.03; 514/419; 514/321; 514/322; 514/438; 514/635; 514/456; 514/657

(57) ABSTRACT

A method of treating Mild Cognitive Impairment has been discovered. The treatment method comprises administering an effective amount of a serotonergic agent, including, but not limited to dexnorfenfluramine. The agent can be any serotonergic agonist, partial agonist, serotonin reuptake inhibitor, or combinations of these agents. The treatment method also encompasses combinations of serotonergic agents and non-steroidal anti-inflammatory agents. The treatment method may also delay the onset of Mild Cognitive Impairment, dementia, or both.

### 2.0 CLAIM OF PRIORITY

The present application is related to, and claims priority from, U.S. Provisional Application No. 60/246,615 filed Nov. 8, 2000, which is incorporated herein by reference in its entirety.

### 1.0 STATEMENT OF FEDERAL SUPPORT

The present invention is made in whole or in part with financial support from the Federal Government under grant NIH #MH-28783. The Federal Government may have rights in the invention.

### 3.0 FIELD OF THE INVENTION

The present invention relates to compositions and methods for treating Mild Cognitive Impairment (MCI) and related symptoms. MCI describes a set of non-disease symptoms that in some cases may lead to Alzheimer's Disease (AD). In fact, persons with MCI are at risk of developing AD, but do not necessarily progress to clinical AD. Currently, no treatments are known to prevent or treat MCI.

### 4.0 BACKGROUND OF THE INVENTION

The social and medical importance of MCI lies primarily in the risk of developing AD or other dementias. AD is the most common neurodegenerative disorder of aging, and is characterized by progressive dementia and personality dysfunction. The abnormal accumulation of amyloid plaques in the vicinity of degenerating neurons and reactive astrocytes is a pathological characteristic of AD. As the fourth leading lethal disease in industrialized societies, surpassed only by heart disease, stroke and cancer, AD affects 5-11% of the population over the age of 65 and 30% of those over the age of 85.

Mild Cognitive Impairment is a condition that is considered to be within the range of normal function. Yet those with MCI are at higher risk of developing AD and, likely, other neurodegenerative diseases. The term Mild Cognitive Impairment is synonymous with Age-Related Memory Impairment, Age-Consistent Memory Impairment, Late-Life Forgetfulness, Age-Related Cognitive Decline, and Relatively Inefficient Memory.

In general, individuals with MCI are normal in their behavior and function with the exception that their memory is poor. Additional and more specific psychometric analyses are used to better define this group.

One operational definition of MCI is a complaint of poor memory and objective evidence of memory performance that is one standard deviation or more below the mean for young adults. This definition uses as a standard the value for “restoration” of memory. Yet another psychometric standard is that the memory of the individual is one standard deviation or more below the mean for the individual's age group. Individuals with MCI generally complain of impaired memory but function normally in ordinary activities of daily living. Moreover, individuals with MCI usually have normal general cognitive functional and are not demented. However, individuals with MCI have an abnormally poor memory for their age.

A number of psychometric measures are useful in the invention. Memory can be tested by any of several methods known in the art, including learning and recall of word lists, paragraphs and/or non-verbal materials. Other useful measures of cognitive function that can be used to determine whether an individual has MCI include, but are not limited to: clinical dementia rating scale, the clinical dementia rating sum of boxes, global deterioration scale, the geriatric depression scale, the mini-mental state examination, the dementia rating scale, the Wechsler Adult Intelligence scale, performance IQ, the Boston naming test, the controlled oral word association test, the logical memory I test, the logical memory II test, visual reproductions test one, visual reproductions test two, the auditory verbal learning test-sum of learning trials 2, the auditory verbal learning test I delayed recall/trial, the free and cued selective reminding test sum of the performance across trials 2 , and the free and cued selective reminding test-delayed recall/trial 6×100. In general, individuals with MCI have psychometric scores on one or more of the above measures that are one standard deviation or more from those of normal controls. The controls can be age- and education-matched.

In contrast, individuals with MCI can have normal psychometric measures using the Wechsler adult intelligence scale—verbal IQ, and the Wechsler adult intelligence scale—full-scale IQ.

By one preferred definition, individuals with MCI have general cognitive measures within 0.5 standard deviations of control subjects and also have memory performance 1.5 standard deviations below control subjects. An objective, documented decline in memory is useful in determining which individuals have MCI.

Individuals with clinically recognizable AD can have psychometric scores that are worse than individuals with MCI or normal controls. Among the psychometric tests useful to distinguish MCI and AD are: the clinical dementia rating scale—sum of boxes, the global deterioration scale, the mini-mental state examination, the dementia rating scale, the Wechsler adult intelligence scale—Verbal IQ, the Weschler adult intelligence scale—full scale IQ, the Wechsler adult intelligence scale—performance IQ, the Boston naming test, the Wechsler memory scale revised—logical memory I, the visual reproductions test one, the visual reproductions test two, the auditory verbal learning test—sum of learning trials 1-5, the auditory verbal learning test-delayed recall/trial 5×100, the free and cued selective remaining test—sum of the performance across trials 1 to 6, and the free and cued selective reminding test—delayed recall/trial 6×100. Individuals with MCI do not meet the criteria for AD. For example, those individuals with MCI can be distinguished from those with AD by a statistically significant difference on a panel of the above-listed psychometric tests. Of note, individuals with MCI have normal general cognitive function. MCI has not been thought to be affected by a serotonergic agent. Thus, methods and compositions to treat MCI with serotonergic agents are unknown.

Amyloid plaques are formed from amyloid precursor protein (APP). APP processing is regulated by neurotransmitters and synaptic activity. Amyloid plaques in AD accumulate near dystrophic neurons and reactive astrocytes. The activation of neurotransmitter receptors, which are coupled to phosphotidylinositol (PI) hydrolysis or to protein kinase C (PKC) activation, can promote APP metabolism and decrease amyloid formation. In contrast to APP which is converted into insoluble deposits known as plaques, a soluble form of APP (APPs) may have beneficial effects. APPs has neurotrophic and neuroprotective functions both in vitro and in vivo. In rats, infusion of soluble APPs improves cognition and synapse density, as well as enhances memory retention in a variety of learning tasks. Activation of neurotransmitters coupled to cAMP production suppresses both constitutive and PKC/PI—stimulated APPs secretion in astroglioma cells and in primary astrocytes. The drastic alterations in neurotransmitter levels and second messenger signaling created by neuro-degeneration and synapse loss in AD may disrupt APP processing in ways that promote the accumulation of amyloidogenic or neurotoxic APP fragments. In contrast, the loss of various neurotransmitters in AD may increase cellular levels of APP holoprotein containing amyloidogenic or neurotoxic peptides due to a decrease in proper APP metabolism.

### 5.0 SUMMARY OF THE INVENTION

The invention is directed to methods of preventing, delaying, attenuating, or ameliorating the symptoms of Mild Cognitive Impairment (MCI) in a subject comprising administering to the subject an effective amount of an agent that stimulates soluble amyloid precursor protein (soluble APP or APPs) secretion, whereby MCI is prevented, delayed, attenuated or ameliorated; and compositions effective therefor. The methods of the invention can be a treatment for subjects with MCI as well a prophylactic treatment for subjects at risk for, but not evidencing MCI. Moreover, the compositions of the invention may be used in combination, and be administered to the subject one or more times each day in any of multiple ways, including orally or parenterally, whereby the subject may be any animal, but preferably a human.

Another aspect of the invention is a method of increasing a level of soluble amyloid precursor protein in the cerebrospinal fluid of a subject comprising administering to the subject an effective amount of an agent that stimulates secretion of soluble amyloid precursor protein into the cerebrospinal fluid, whereby the level of soluble amyloid precursor protein in the cerebrospinal fluid is increased. The level of soluble amyloid precursor protein can be determined by direct assay of cerebrospinal fluid, such as can be obtained by a spinal tap, or by any indirect method that measures a behavior, a function, or a factor, the concentration of which factor is highly correlated to the level of soluble APP.

Another aspect of the invention is a method of preventing, attenuating or ameliorating the symptoms of Mild Cognitive Impairment in a subject comprising administering to the subject an effective amount of a serotonergic agent that stimulates soluble amyloid precursor protein secretion, whereby the impairment is prevented, attenuated or ameliorated. Another aspect of the invention is a method of preventing, delaying, attenuating or ameliorating the symptoms of Mild Cognitive Impairment in a subject comprising administering to the subject an effective amount of a serotonergic agent that stimulates soluble amyloid precursor protein secretion, in which the agent is not a non-steroidal anti-inflammatory agent. The serotonergic agent may be a serotonergic agonist, a partial agonist or serotonin reuptake inhibition.

Another aspect of the invention is a method of increasing the level of APPs in the cerebrospinal fluid of a subject comprising administering to the subject an effective amount of a serotonergic agent that stimulates secretion of APPs into the cerebrospinal fluid, whereby the level of APPs in the cerebrospinal fluid is increased.

Yet another aspect of the invention is a method of decreasing the level of APPs in the cerebrospinal fluid comprising administering an effective amount of a serotonin antagonist.

These and other objects of the invention will be evident to those of ordinary skill from a consideration of the discussions and descriptions provided in this specification, including the detailed description of the preferred embodiments. It is to be understood that both the foregoing general description and the following detailed description are exemplary and are intended to provide further explanation of the invention claimed.

### 6.0 BRIEF DESCRIPTION OF THE DRAWINGS

The accompanying drawings, which are incorporated in and constitute part of this specification are included to illustrate and provide a further understanding of the methods and compositions of he invention. Together with the description, the drawings serve to explain the principles of the invention.

FIG. 1 depicts a dose response to dexnorfenfluramine on APPs levels in CSF.

FIG. 2 depicts the inhibition of dexnorfenfluramine induced APPs secretion into CSF by ritanserin or ketanserin.

FIG. 3 depicts a time course of dexnorfenfluramine effect on APPs levels in CSF.

FIG. 4 depicts the effect of chronic administration of dexnorfenfluramine on APPs levels in the cerebrospinal fluid of guinea pigs.

### 7.0 DETAILED DESCRIPTION OF THE EMBODIMENTS

One embodiment of the invention encompasses a method of preventing, delaying, attenuating, or ameliorating the symptoms of MCI in a subject at risk thereof by administering an effective amount of a composition comprising a substance that increases soluble amyloid precursor protein expression in the subject. The composition of this invention can be a serotonin precursor or prodrug, a serotonergic agonist, a serotonergic partial agonist, a serotonin reuptake inhibitor, an inhibitor of serotonin degradation, a stimulator of serotonin synthesis, or combinations thereof. The composition may be administered at least one to several times per day, using any of several methods of administration known in the art, such as orally or parenterally, and the subject can be any animal, preferably a human. The agents are effective at a wide range of doses, and can differ based on the characteristics of the particular agent. In one aspect, the dose may be at least about 0.1 mg to about 1000 mg/kg bodyweight per day. In another aspect the dose is at least about 1 mg to about 500 mg/kg bodyweight per day. In another aspect the effective amount is at least about 10 mg to about 200 mg/kg bodyweight per day. In one aspect, the daily dose is between about 50 to about 100 mg/kg body weight per day.

The applicants also disclose a method of treating the symptoms of Mild Cognitive Impairment in a subject in need comprising administering an effective amount of a modified serotonergic agent, wherein the modified agent increases soluble amyloid precursor protein expression in the subject, wherein the increase in soluble amyloid precursor protein expression alleviates the symptoms of the MCI.

Without being limited to a mechanism of action, the compositions of the invention comprise serotonergic agents that increase secretion of soluble amyloid precursor protein. Soluble amyloid precursor protein has neuroprotective properties, increases neuronal outgrowth and regeneration, promotes synapse formation, and enhances cognition, learning, and memory. Some of the compositions of the invention can bind to serotonin receptors and transporters, and thereby regulate soluble APP secretion and cell-associated levels of APP holoprotein. Some of the compositions of the invention can bind to other neurotransmitter receptors and transporters, and indirectly modulate serotonin levels or efficacy. By these or other means, the agents can alter APP synthesis, APP expression, α-APP production, and soluble APP (APPs) secretion.

The compositions of the invention comprise of serotonin, serotonin precursors, serotonin agonists, serotonin degradation inhibitors, serotonin reuptake inhibitors, or combinations thereof.

The invention also encompasses a method to inhibit excessive amyloid formation, prevent neurite dystrophy and alleviate pathological symptoms, such as neurodegeneration or cognitive deficits that may arise from the negative effects of inappropriately expressed, produced, or formed amounts of APP, and which may be associated with loss of cognition.

It is also an object of the invention to prevent the debilitating effects of injury or trauma to the brain, as well as ameliorate or delay early stage neurological diseases and neurodegenerative disorders, such as Alzheimer's, Parkinson's, or Lou Gehrig's Disease (amylotrophic lateral sclerosis), multiple sclerosis and the like, which may have their roots in the formation or presence of amyloid plaques.

Serotonergic Agents. In one aspect, the invention provides a method of preventing, attenuating or ameliorating the symptoms of MCI in a subject comprising administering to the subject an effective amount of an agent that stimulates soluble amyloid precursor protein secretion, in which the agent is not a non-steroidal anti-inflammatory agent. The subject can be in need of such a treatment. Serotonin agonists, partial agonists, serotoninergic reuptake inhibitors, and combinations thereof, are found to be effective to alleviate the symptoms of MCI. In a particular embodiment, the serotonergic agents can be combined with non-steroidal antiinflammatories. The agent(s) can be administered in a pharmaceutically acceptable carrier.

In one aspect, the invention relates to a method of increasing a level of APPs in the cerebrospinal fluid (CSF) of a subject comprising administering to the subject an effective amount of an agent that stimulates secretion of APPs into the cerebrospinal fluid, whereby the level of APPs in the cerebrospinal fluid is increased. The subject can be in need of such a treatment. The agent effective in increasing APPs in the CSF can be a serotonergic agonist, partial agonist, selective serotonin reuptake inhibitor, or combinations thereof.

In another aspect, the invention relates to a method of decreasing a level of APPs in the cerebrospinal fluid comprising administering an effective amount of a serotonin antagonist. The level of APPs suitable for treatment with a serotonin antagonist can be an abnormally high level. The subject can be in need of such treatment or the treatment can be part of an experimental model. The effective amount of antagonist can be between about one microgram per kilogram body weight and about 10 milligrams per kilogram body weight. The effective amount of antagonist can be between about 0.1 milligram per kilogram body weight and 5 milligrams per kilogram body weight.

The invention is not limited by terms used in the art and is also described by psychometric criteria.

The agents of the invention are effective at preventing and treating MCI. In addition, the agents are effective at enhancing synapse formation. Thus, the agents can be used to ameliorate the consequences of cerebral ischemia. The cerebral ischemia may be acute or chronic and includes transient ischemic accidents, head trauma, and stroke.

Cell-associated APP holoproteins also known as Abeta (or Aβ) are associated with loss of cognition, poor memory, and AD.

The inventors have observed a link between features associated with MCI and the function of serotonin receptors. A number of compounds are known to stimulate or enhance serotonin-mediated neurotransmission and are sometimes referred to as serotoninergic drugs but a more standard usage is “serotonergic drugs” or “serotonergic agents.” All serotonergic agonists are selective serotonin reuptake inhibitors (SSRIs) and are considered effective agents in the invention for administration to subjects exhibiting symptoms of MCI. Examples of these compounds, for purposes of illustration and not limitation, include the following: dexnorfenfluramine, dexfenfluramine, fenfluramine, sertraline, tryptophan, 5-hydroxytryptophan, clomipramine, fluoxetine, paroxetine, fluvoxamine, citalopram, femoxitine, cianopramine, sertraline, sibutramine, venlafaxine, ORG 6582, RU 25591, LM 5008, DU 24565, indalpine, CGP 6085/A, WY 25093, alaprociate, zimelidine, trazodone, amitriptyline, imipramine, trimipramine, doxepin, protriptyline, nortriptyline, dibenzoxazepine, deprenyl, isocarboxazide, phenelzine, tranylcypromine, furazolidone, procarbazine, moclobemide, brofaromin, nefazodone, bupropion, MK 212, DOI, m-CPP, Ro 60-0175/ORG 35030, Ro 60-0332/ORG 35035, Ro 60-0175, Org 12962, Ro 60-0332, methyl-5-HT, TFMPP, bufotenin, Ru 24969, quipazine, 5-carboxyamidotryptamine, sumatriptan, CGS 12066, 8-OH-DPAT, (S)-2(chloro-5-fluoro-indol-1-yl)-1 -methylethylamine 1:1 C4H4O4, (S)-2-(4,4,7-trimethyl-1,4-dihydro-indeno(1, 2-b)pyrrol-1-yl)-1-methylethylamine 1:1 C4H4O4, SB 206553, and pharmaceutically acceptable salts thereof. Other examples of serotoninergic drugs that can directly or indirectly affect MCI include 2-aminomethyl-chromans; 2,3-Dihydro-1H-inden-1-yl)ethyl-4-(naphthalen-1-yl)piperazines; polypeptides having a serotonin receptor activity; 4-amino-1-(2-pyridyl)piperidines; 1-heteruarylazetidines and pyrrolidines; 4-aminomethylpiperidines; 2-[3-(3-indolyl) 2-amino propionyloxy] acid salts; dipeptides of L-5hydroxytryptophan; C-Homo-9-oxa-ergolines; 9-Oxalysergic acid derivatives; dopaminergically stimulating 4-substituted indoles; 2-aminoindan compounds; N-aryl piperidine compounds; benzisothiazolyl-substituted aminomethylchromans; azaheterocyclymethyl-chromans; 1-halopyrazin- or 1-halopyrimidin-4-amino-4-alkylpiperidines; 3-aminochroman compounds; 1-halopyridin-4-amino-4-alkylpiperidines; alkoxy-3-[(toluenesulfonylaminoalkyl)amino] chroman compounds; piperidylmethyl-substituted chroman derivatives; benzoxazinone compounds; 1(mono- or bis(trifluoromethyl)-2-pyridinyl)piperazines; 2-methyl-serotonin; methysergide; ICS 205-930; zacopride; naltrexone; nalmefene; (+)-alpha-(2,3-dimethoxyphenyl)-1-[2-(4-fluorophenyl) ethyl]-4-piperidinemethanol; S-adenosyl methionine; N—acetyl serotonin; bicyclolactam compounds; neuropeptide Y/peptide YY agonist and antagonists; ratite extracts; substituted morpholine derivatives; galanin receptor (GALR2) antagonist; 3-piperidinyl-substituted 1,2-benzisoxazoles and 1,2-benzisothiazoles; ondansetron; indanol compounds; 2-imidazoline, 2-oxazoline, 2-thiazoline, and 4-imidazole derivatives of methylphenyl, methoxyphenyl, and aminophenyl alkylsulfonamides and ureas; quinoline leukotriene antagonists; heteroaryl spiroethercycloalkyl tachykinin receptor antagonists; morpholine and thiomorpholine tachykinin receptor antagonists; NMDA receptor antagonists; aminoalkylindoles; indole-ethanamines; tetrahydro-beta-carbolines; purinylalkyl benzamide derivatives; hydantoin; sinemet; amantadine; symmetrel; 1-amino-adamantane; bromocriptine mesylate; pergolide mesylate; selegiline; benztropine mesylate; trihexylphenidyl; procyclidine; biperiden; and ethopropazine among many others.

Serotonin-mediated neurotransmission can also be enhanced by administering drugs, such as quipazine, m-CPP, MK212 or CM57493, which activate post-synaptic serotonin receptors. The chemical names of DU 24565, CGP 6085/A, and WY are, respectively, 6-nitroquipazine, 4-(5,6-dimethyl-2-benzofuranyl) piperidine HCl, and 1-[1-([indol-3-yl]methyl) piperid-4-yl]-3-benzoylurea, respectively. For additional details, see for example U.S. Pat. Nos. 5,179,126; 5,223,540; 4,999,382; or 4,971,998 incorporated herein by way of reference.

When the quantity of serotonin is aberrant at a given time or over a period of time, patients can be administered a drug which has any of the following effects: increases serotonin production (e.g., tryptophan lithium); causes serotonin release, e.g., d-fenfluramine, d,1-fenfluramine, d-norfenfluramine or d,1-norfenfluramine; suppresses serotonin reuptake, e.g., fluoxetine, fluvoxamine, citalopram, chlorimipramine (also known as clomipromine) femoxetine, cianopramine, ORG 6582, RU 25591, LM5008, sertraline or 1S-4S-N-methyl-4-(3,4 dichlorophenyl)-1,2,3,4,-tetrahydro-1-naphthylamine, paroxetine, DU 24565, indalpine, CGP 6085/A, WY 25093, alaprociate, zimelidine, cyanimipramine, desyrel (trazodone hydrochloride) or trazodone amitriptyline or elavil (amitriptyline hydrochloride), imipramine or tofranil (imipramine hydrochloride), trimipramine or surmontil, doxepin or sinequan (doxepin hydrochloride), protriptyline or vivactil (protriptyline hydrochloride), nortriptyline or aventyl (nortriptyline hydrochloride), dibenzoxazepine (also known as amoxapine or asendin); blocks presynaptic receptors, e.g., metergoline, methysergide, cyproheptadine (which can also block postsynaptic receptors); or blocks monoamine oxidase, e.g., deprenyl, marplan or isocarboazide, nardil (phenelzine sulfate) or phenelzine, parnate (tranylcypromine sulfate) or tranylcypromine, furazolidone, procarbazine, moclobemide or aurorix, brofaromine). One skilled in the art will recognize that in certain situations serotonin is not directly involved but may act via another neurotransmission pathway. For example drug compounds used for manic and manic depressive disorders such as lithium, tricyclic compounds (imipramine, amitriptyline, trimipramine, doxepin, desipramine, nortriptyline, protriptyline, amoxapine, clomipramine, maprotriline, and carbamazepine) and antidepressants including, but not limited to bupropion, sertraline, fluoxetine, and trazodone inhibit not only serotonin but also norepinephrine reuptake. Thus, the preferred group of patients to be treated by the compositions of this invention are not necessarily limited to treatment with the serotonergic agents.

The loss of synapses has been suggested to be an early event in the pathology of AD, and appears to be related to the extent of reactive astrogliosis. The loss of synapses may be a component of MCI as well. The invasion and proliferation of reactive astrocytes within these regions of degeneration may explain the increased levels of glial fibrillary acific protein (GFAP) in the brain tissue and cerebrospinal fluid of AD. Indeed, the upregulation of β-adrenergic receptors in the frontal cortex and hippocampus of AD brains has been attributed to the proliferation of astrocytes associated with neurodegeneration. Circulating levels of norepinephrine after brain injury appear to cause reactive astrogliosis and cell proliferation. The aberrant activation of β-adrenergic receptors coupled to cAMP signaling by norepinephrine might also stimulate APP overexpression in astrocytes.

APP overexpression in cultured astrocytes treated with PG E₂ is associated with the secretion of APP holoprotein. Although secreted APP is usually truncated at the C-terminus, antisera C8 which is directed at the C-terminus of APP detects increased amounts of APP holoprotein (˜130 kD) in the media of astrocytes treated with PG E₂ for 24 h. The disclosure of the present invention is consistent with the observation that Chinese hamster ovary cells transected with full-length APP751 cDNA also secrete soluble APP holoprotein. APP holoprotein can be detected in the cerebrospinal fluid of humans, and can be actively released from secretory vesicles in response to receptor stimulation or neuronal depolarization. It is not known if secreted APP holoprotein is reinternalized for subsequent processing, or if it can be metabolized in the extracellular space.

Thus, the present studies show that stimulation of serotonin increases the production of neuroprotective soluble APP. The upregulation or activation of serotonin receptors in brain regions that are vulnerable to damage can stimulate synapse formation, inhibit amyloid production and inhibit APP overexpression. Because APP overexpression can cause cognitive dysfunction, the inventors have shown that such substances as dexnorfenfluramine (also known as (+)Norfenfluramine and (+)2-amino-1-(3-trifluoromethylphenyl) propane) are useful drug candidates for the treatment of symptoms of Mild Cognitive Impairment.

7.1. Further Aspects of the Embodiments

It should be apparent that in one aspect, the present invention is directed to a method of alleviating the negative effects of a pre-disease state stemming from the aberrant expression, production, or formation of amyloid precursor protein (APP) in a subject.

7.2. Compositions of the Present Invention

As should be apparent, the present invention also contemplates compositions comprising the active substances disclosed herein. Preferably, these compositions include compositions comprising a therapeutically effective amount of one or more of the active compounds or substances along with a pharmaceutically acceptable carrier.

The carrier can be a food or a nutriceutical carrier. The carrier can be in the form of a powder for preparing a beverage, a lozenge or dissolving candy, a chewable food, a topping such as croutons or a salad dressing, or a prepared beverage.

The carrier means a non-toxic, inert solid, semi-solid liquid filler, diluent, encapsulating material, formulation auxiliary of any type, or simply a sterile aqueous medium, such as saline. Some examples of the materials that can serve as carriers are foodstuffs, sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, alpha-tocopherol and the like; and the metal chelating agents such as citric acid, ethylenediamine tetraacetic acid (EDTA), sorbitol, tartaric acid, phosphoric acid and the like.

By “effective amount” of an active compound, such as dexnorfenfluramine is meant a sufficient amount of the compound to prevent, delay, ameliorate or alleviate the negative effects of a predisease condition stemming from the aberrant expression, production, or formation of amyloid precursor protein (APP) at a reasonable benefit/risk ratio applicable. The specific effective dose level for any particular individual will depend upon a variety of factors including the severity of the pre-disease condition, activity of the specific compound employed; the specific composition employed; the age, body weight, general health, sex and diet of the subject; the time of administration, route of administration, and rate of excretion of the specific compound employed; the duration of the treatment; drugs used in combination or coinciding with the specific compound employed; and like factors well known in the medical and nutritional arts.

The total daily dose of the compounds of the present invention administered to a subject in single or in divided doses can be in amounts, for example, from about 0.0001 to about 25 mg/kg body weight or more usually from about 0.01 to about 15 mg/kg body weight. Single dose compositions may contain such amounts or submultiples thereof to make up the daily dose. In general, treatment regimens according to the present invention comprise administration to a human or other mammal in need of such treatment from about 1 mg to about 1000 mg of the active substance(s) of this invention per day in multiple doses or in a single dose of from 1 mg, 5 mg, 10 mg, 100 mg, 500 mg or 1000 mg.

In certain situations, it may be important to maintain a fairly high dose of the active agent in the blood stream of the patient, particularly early in the treatment. Hence, at least initially, it may be important to keep the dose relatively high and/or at a substantially constant level for a given period of time, preferably, at least about six or more hours, more preferably, at least about twelve or more hours and, most preferably, at least about twenty-four or more hours.

The compounds of the present invention may be administered alone or in combination or in concurrent therapy with other agents which affect the central or peripheral nervous system, particularly selected areas of the brain.

Liquid dosage forms for oral administration may include pharmaceutically acceptable emulsions, microemulsions, solutions, suspensions, syrups and elixirs containing inert diluents commonly used in the art, such as water, isotonic solutions, or saline. Such compositions may also comprise adjuvants, such as wetting agents; emulsifying and suspending agents; sweetening, flavoring and perfuming agents. For example the agents can be administered in flavored drinks, including fruit juice combinations and vanilla or chocolate-flavored milk-based drinks.

Solid dosage forms for oral administration may include liquid or chewable foodstuffs, capsules, tablets, pills, powders, gelcaps and granules. Suitable foodstuff forms are candy bars, granola bars, “power” bars, and fruit bars. Other suitable foodstuff forms are beverages or powders to be mixed into beverages. In such solid dosage forms the active compound may be admixed with at least one inert diluent such as a foodstuff, sucrose, lactose or starch. Such dosage forms may also comprise, as is normal practice, additional substances other than inert diluents, e.g., tableting lubricants and other tableting aids such as magnesium stearate and microcrystalline cellulose. In the case of capsules, tablets and pills, the dosage forms may also comprise buffering agents. Tablets and pills can additionally be prepared with enteric coatings and other release-controlling coatings.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferably, in a certain part of the intestinal tract, optionally in a delayed manner. Examples of embedding compositions which can be used include polymeric substances and waxes.

Dosage forms for topical or transdermal administration of a compound of this invention further include ointments, pastes, creams, lotions, gels, powders, solutions, sprays, inhalants or patches. The active component is admixed under sterile conditions with a acceptable carrier and any needed preservatives or buffers as may be required. Ophthalmic formulations, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the active compounds of this invention, excipients such as lactose, talc, silicic acid, aluminum hydroxide, calcium silicates and polyamide powder, or mixtures of these substances. Sprays can additionally contain customary propellants, such as chlorofluorohydrocarbons.

Transdermal patches have the added advantage of providing controlled delivery of active compound to the body. Such dosage forms can be made by dissolving or dispersing the compound in the proper medium. Absorption enhancers can also be used to increase the flux of the compound across the skin. The rate can be controlled by either providing a rate controlling membrane or by dispersing the compound in a polymer matrix or gel.

Accordingly, the present invention is useful in the treatment or alleviation of pre-disease states, especially MCI, transient ischemic accidents, hemorrhagic stroke and occlusive stroke to name a few and central or peripheral nervous system damage, dysfunction, or complications involving same stemming from edema, injury, or trauma. Such damage, dysfunction, or complications may be characterized by an apparent neurological, physiological, psychological, or behavioral aberrations, the symptoms of which can be reduced by the administration of an effective amount of the active compounds or substances of the present invention.

Still other therapeutic “strategies” for preventing an immune or inflammatory reaction can be adopted including, but not limited to, cell/tissue transplantation, gene and stem cell therapy, adjuvant therapy, extracorporeal therapy; use of telerogenic peptides, plasmapheresis and immunoadsorption.

In addition the compositions of the invention can be used in combination with other NSAIDS, neurotransmitter agonists, antagonists, and modulators. In a particular example, an effective amount of dexnorfenfluramine or other serotonergic agent can be administered to a subject having MCI, in combination with an effective amount of adenosine, adrenoreceptors, angiotensin, atrial naturiuretic peptide, bombesin, bradykinin, cholecystokinin, gastrin, dopamine, endothelin, GABA, glutamate, histamine, interleukin-1, leukotriene, acetylcholine, carbachol, neuropeptide Y, nicotinic acetylcholine, opioid, platelet activating factor, prostanoid, purinoceptors, somatostatin, tachykinin, thrombin, vasopressin, oxytocin, vasoactive intestinal peptide, and the like. In a more particular example, the compositions of the invention, for example dexnorfenfluramine, can be administered in combination with other memory-promoting agents, including, but not limited to, acetylcholinesterease inhibitors, CDP-choline, uridine, uridine/choline or other neurotransmitter receptor agonists.

The following examples are provided for further illustration of the present invention, and do not limit the invention.

### 8.0 EXAMPLES

Experiments and exemplary procedures are described below which provide additional enabling support for the present invention. In particular, in vitro studies using primary cultures of rat cortical astrocytes and in vivo studies using appropriate animal models are disclosed.

8.1. General Methods

Astrocytes are isolated from cortices from postnatal rats by methods standard in the art. In brief, dissected cortices are dissociated by trypsinization and trituration through a flamepolished Pasteur pipette. Cells are plated onto poly-L-lysine coated 35- or 100 mm culture dishes at densities of about 10-25 cells/mm². The initial culture media, minimal essential medium (MEM, Gibco) containing 10% horse serum (BioWhittaker), are aspirated after 2-5 h after plating to remove unattached cells and debris, and replaced with MEM containing 7.5% fetal bovine serum (FBS, BioWhittaker). Half the media is replaced with MEM/7.5% FBS twice weekly. Cells are kept at 37° C. in a humidified 5% CO₂/95% air incubator. Media are changed twice weekly. Immunocytochemical staining with antibodies against GFAP and tau shows that >90% of cultured cells are astrocytes and <5% are neurons. Pharmacological manipulations are performed in serum-free media on 7-14 DIV confluent astrocytes.

8.2. Detection of Cell-Associated Protein

To detect cell-associated proteins (APP or GFAP), astrocytes from 35 mm dishes are scraped into lysis buffer (60 mm Tris/HCL 4% SDS, 20% glycerol, 1 mm dithiothreitol), ultrasonicated and boiled for 5 mm. The total amount of cell protein per dish, estimated using the bicinchoninic acid assay, is not altered by pharmacological treatments. Bromphenol blue (0.1%) is added to each sample and equal amounts of protein (75 mg/lane) are loaded on 10% SDS-polyacrylamide gels.

To detect secreted APP, culture media is collected after drug treatments and phenylmethylsulfonyl fluoride is added to a final concentration of 2 mM. The media samples are then applied to Sephadex PD-b desalting columns (Pharmacia) and eluted with distilled water. Column eluates are frozen and dried by vacuum centrifugation. The lyophilized proteins are reconstituted in 25 μL water followed by 25 L of 2X Laemmli gel loading buffer, and boiled for 5 min.

The amount of media or cell protein loaded for sodium dodecyl sulfate-polyacrylamide gel electrophoresis (10-20% SDS PAGE; Bio-Rad) is normalized for the amount of protein per sample. Proteins (equivalent to 100 μg cell protein/lane) are separated by electrophoresis, electroblotted onto polyvinylidene difluoride membranes (Immobilon-P, Millipore) and blocked in Tris-buffered saline with 0.15% Tween 20 (TEST) containing 5% powdered milk for 30 mm. After 2×10 mm rinses in TBST, the membranes are incubated in TEST containing an appropriate antibody. Monoclonal antibodies 22C11 and GFAP (both from Boehringer Mannheim) are used to detect the N-terminus of APP and glial fibrillary acidic protein respectively; antisera R37 and R98 (gifts of Dr. F. Kametani, Tokyo Institute of Psychiatry) are used to detected the C-terminus and KPI motifs of APP respectively; antiserum C8 (gift of Dr. D. Selkoe, Women's Hospital, Harvard Medical School, Cambridge, Mass.) is used to detect the C-terminus of APP.

After an overnight incubation, membranes are rinsed in TBST before being treated for lh with a peroxidase-linked secondary antibody. After several rinses in TEST, protein bands are visualized on Kodak X-AR films by an enhanced chemiluminescence method (Amersham). Optical densities of the protein bands are quantitated by laser scanning densitometry (LICE, Bromma, Sweden), and normalized to the densities of those bands generated under control conditions.

8.3. cAMP Assay

Levels of cyclic AMP are measured with a 8-[³H]-cAMP assay kit (Amersham TRK 432) in astrocytes grown on 35 mm dishes. In brief, after aspirating the medium and rinsing twice with 1 ml ice cold PBS, the cells are scraped in 0.8 ml ice cold ethanol and sonicated. The cell suspension is incubated for 5 min at room temperature, centrifuged and the supernatant is dried in a rotary evaporator. After resuspension in 120 μl Tris/EDTA buffer, two duplicate samples of 50 μL each are mixed with the binding protein, 8-[³H] adenosine 3′, 5′-cyclic phosphate tracer and incubated at 2-4° C. for 2 h. A charcoal suspension (100 μL) is added to the samples before centrifugation and 200 μL of the supernatant is removed for scintillation counting. The amount of cyclic AMP (pmol/mg protein) is estimated by comparing to known standards, and normalized to the amounts of whole cell protein as determined by the bicinchoninic acid assay (Sigma).

8.4. Data Analysis

Measurements of cellular and secreted proteins, or of mRNA in treatment groups are normalized against those of control groups which are prepared in parallel and loaded onto the same blot. Analysis of variance (ANOVA) and t-tests are used to evaluate differences between groups (significance level, p=0.05), using drug treatments as the independent variable

8.5. Analysis of RNA

Total RNA from astrocytes grown on 100 mm dishes is extracted by the acid guanidium thiocyanate-phenol chloroform method. In brief, the medium is aspirated and the cells are scraped in 1 ml of TRI Reagent. After incubation for 15 min at room temperature, 0.2 ml chloroform is added, mixed vigorously with TRI Reagent and the mixture is stored for another 15 min at room temperature. After centrifugation at 12,000 g for 15 min, 0.5 ml isopropanol is added to the aqueous phase of the mixture to precipitate RNA. The RNA pellet collected by centrifugation (12,000 g, 15 min at 4° C.) is washed with 70% ethanol once and solubilized in an appropriate amount of Formazol (Molecular Research Center, Cincinnati, Ohio). RNA samples (20 μg) are denatured by heating for 15 mm at 60° C. prior to loading onto 1.2% agaroseformaldehyde gels for electrophoresis. RNA is blotted onto Hybond polyvinyl membranes by overnight capillary transfer and fixed onto the membranes by uv light illumination. Membranes are pre-hybridized with Amersham Rapid-hyb (Amersham Lab, Arlington Heights, Ill.) buffer for 2 h and labeled overnight with a -p1.8 kb human APP cDNA (gift of Dr. Rachael Neve, McLean Hospital, Harvard Medical School, Belmont, Mass.) or human glyceraldehyde-3-phosphate dehydrogenase probe (G3PDH; Clontech) labeled with [³²P]dCTP using random primed extension (Amersham Megaprime DNA labelling kit). Membranes are dried and exposed to Kodak X-ray film for 24-48 h with an Amersham enhancer sheet. The relative amounts of mRNA obtained by hybridization are estimated using densitometric analysis of autoradiographs. The levels of APP MRNA are normalized to the amounts of G3PDH mRNA and expressed as a ratio to the levels of untreated, control cells.

8.6. In Vivo Studies

The present studies indicate that serotonergic agents, including dexnorfenfluramine, can inhibit APP overexpression in GFAP-immunoreactive cultured astrocytes. Reactive astrocytes (that is, astrocytes that have been activated or stimulated in some fashion, e.g., those associated with brain or neuronal injury) in vivo also upregulate GFAP expression. Indeed, the examination of post-mortem brains in patients with AD shows that reactive astrocytes are found in proximity to amyloid plaques and regions of neurodegeneration. The inventors believe that neuronal, brain, or head injury gives rise to the formation of reactive astrocytes, which overexpress APP and contribute to the formation of amyloid or neurotoxic APP derivatives, which may contribute to MCI. Thus, animal models of head injury exhibit increased amounts of APP in the brain.

8.7. Materials and Methods Related to the Action of Serotoneraic Agents on APPs in the CSF

Guinea pigs are acclimatized to animal housing conditions for at least 70 days before receiving drug treatments or CSF withdrawal. CSF is withdrawn from the cisternae magna. Briefly, the dorsal cervical area and the occipital area of the skull is shaved and cleansed with alcohol after anesthesizing the guinea pigs with ketamine. A 33-gauge needle is used to penetrate the skin, the atlanto-occipital membrane and the dura mater in the region between the anterior of the first vertebra, and posterior of the cerebellum and the occipital protuberance. Upon penetration of the cistemae magna, a slight resistance in advancing the needle is felt; CSF is withdrawn via a PE10 polyethylene tubing attached to the needle at one end, and to a 20 ml syringe at the other. No more than 200 μL of CSF is withdrawn from each guinea pig per session or within a 24h interval. Occasionally, blood is present in the CSF; these samples are discarded and not used for analysis. The entire procedure for CSF withdrawal is completed within 5 minutes.

Guinea pigs are randomly assigned to receive i.p. injections of either 0.5, 1.0, 2.0 or 4.0 mg/kg dexnorfenfluramine with or without ritanserin (1 mg/kg) or ketanserin (2 mg/kg); CSF is withdrawn two hours after drug treatments. Sterile normal saline is given control guinea pigs. To determine the time-course of APPs secretion, CSF is withdrawn at 1, 2, 4 or 8h after i.p. injections of either 1 mg/kg dexnorfenfluramine or saline. To determine the effect of chronic dexnorfenfluramine administration on secreted and brain APP levels, guinea pigs are injected with 2 mg/kg dexnorfenfluramine for 10 consecutive days. Withdrawal of CSF is performed 2h after dexnorfenfluramine on the tenth day, after which the guinea pigs are sacrificed and brains removed for analysis. Each guinea pig is repeatedly and randomly assigned on different days for either dose-response, for time-course, and for dexnorfenfluramine-5HT antagonist studies of APPs or Aβ secretion.

CSF (1-200 μL) is used for Western blot analysis of secreted APPs. For analysis of APP holoprotein, brain tissue from the cortex and hippocampus are collected in Eppendorf tubes and sonicated in 50 μL lysis buffer (60 mM Tris-HCl, 4%SDS, 20% glycerol, 1 mM dithiotreitol). The samples are boiled for 10 min to inhibit protease activity. The total amount of protein in each sample is measured by the bicinchoninic acid (Sigma) assay. Prior to gel electrophoresis, 1 μL of 5% bromphenol blue solution is added to each sample.

The amount of CSF or cell protein loaded for sodium dodecyl sulfate-polyacrylamide gel electrophoresis (10-20% SDS PAGE; Bio-Rad) is normalized for the amount of protein per sample. Proteins (equivalent to ˜100 μg cell protein/lane) are separated by electrophoresis, electroblotted onto polyvinylidene difluoride membranes (Immobilon-P, Millipore) and blocked in Tris-buffered saline with 0.15% Tween 20 (TBST) containing 5% powdered milk for 30 min. After 2×10 min rinses in TBST, the membranes are incubated in TBST containing an appropriate antibody. Monoclonal antibody 22C11 (from Boehringer-Mannheim) and APP-KPI (from Chemicon) were used to detect the N-terminus and KPI-domain of APP respectively; an antibody against glial fibrillary acidic acid (GFAP) is obtained from Chemicon.

After an overnight incubation, membranes are rinsed in TBST before being treated for lh with a peroxidase-linked secondary antibody. After several rinses in TBST, protein bands are visualized on Kodak X-AR films by an enhanced chemiluminescence method (Amersham). Optical densities of the protein bands are quantitated by laser scanning densitometry (LKB, Bromma, Sweden), and normalized to the densities of those bands generated under control conditions.

Measurements of soluble Aβ₁₋₄₀ or Aβ₁₋₄₂ in the CSF are quantified using the Aβ40 or Aβ42 enzyme-linked immunosorbent assay (ELISA) kit (QBC, Hopkinton, Mass.) using the procedures recommended by the manufacturer.

Data Analysis—Measurements of cellular and secreted proteins are normalized against those of control groups prepared in parallel and loaded onto the same blot. Analyses of variance (ANOVA) and t-tests for repeated measures are used to evaluate differences between groups (significance level, p=0.05), using drug treatments as the independent variable. Data are presented as means±SE, n=number of independent experiments.

8.8. The Effect of Dexnorfenfluramine on APPs in CSF

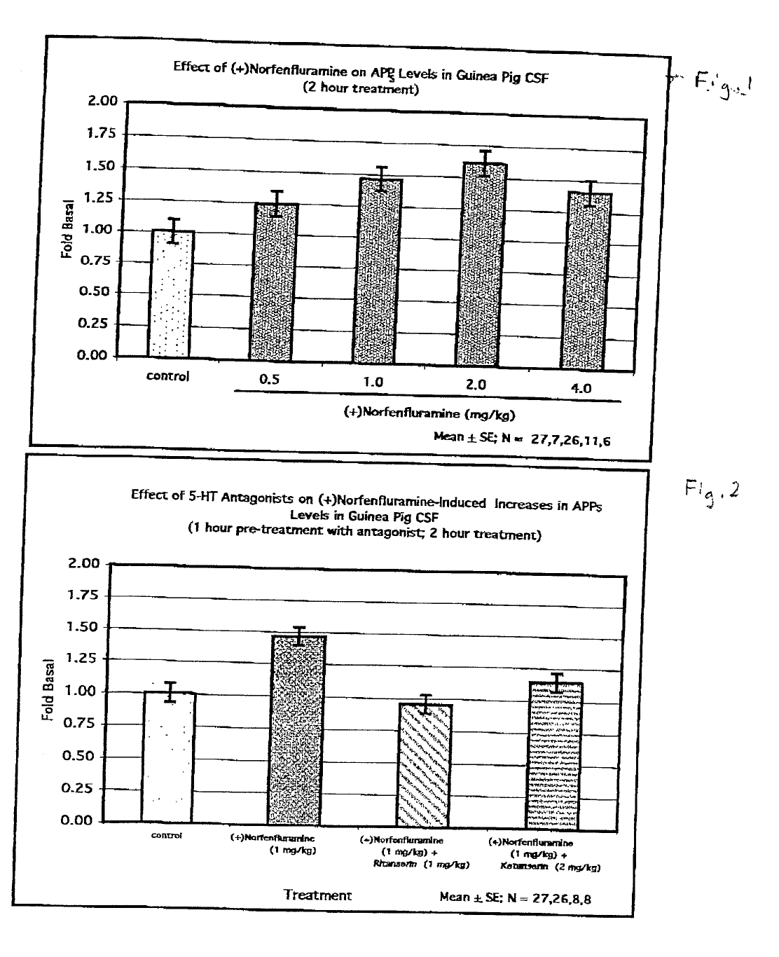

Intraperitoneal injections of guinea pigs with 0.5, 1, 2 or 4 mg/kg dexnorfenfluramine are used to produce APPs levels in the cerebrospinal fluid that are 1.25-, 1.45-, 1.6- and 1.4-fold respectively, relative to those levels of saline-injected, control animals (all p<0.05) (FIG. 1). These increases in APPs secretion are observed in the CSF one hour after i.p. injections with 0.5, 1, 2 or 4 mg/kg dexnorfenfluramine. The stimulatory effect of dexnorfenfluramine (1 mg/kg) on APPs is completely inhibited by the serotonin antagonist ritanserin (1 mg/kg) or ketanserin (2 mg/kg) (FIG. 2), as revealed by Western blot analysis of CSF samples withdrawn one hour after injections. The levels of APPs in the CSF at 1h, 2h, 4h or 8h after injections with 1 mg/kg dexnorfenfluramine, are examined and the levels are found to show that APPs levels are 1.5-, 2.0-, 1.3- and 1.5-fold respectively, relative to the level of the control group (FIG. 3). Injections with 0.5, 1, 2 or 4 mg/kg dexnorfenfluramine is found to have no significant effect on the levels of Aβ1-40 or of Aβ1-42 in the cerebrospinal fluid relative to the control group (p>0.05).

8.9. Chronic Administration of Dexnorfenfluramine

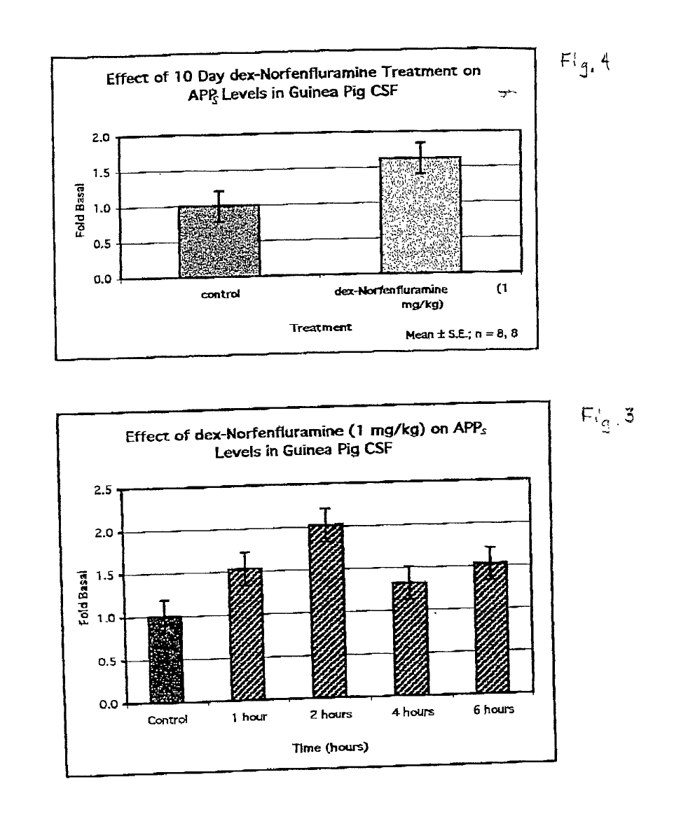

The levels of APP holoprotein, Aβ1-40, and Aβ1-42 in the brain are analyzed after 10 consecutive days of i.p. injections with dexnorfenfluramine (1 mg/kg), as were the levels of APPs, Aβ1-40 and Aβ1-42 in the CSF. Brain APP holoprotein levels are not altered by dexnorfenfluramine (p>0.05). Analysis of CSF indicated that APPs levels are increased by 1.6-fold (FIG. 4), and both Aβ1-40 and Aβ1-42 levels are decreased by administration of dexnorfenfluramine relative to the levels of control groups.

8.10. Use of Serotonergic Agents for Treatment of MCI

In one aspect of the invention, the agents dexnorfenfluramine, dexfenfluramine, fenfluramine, fluoxetin, sertraline, paroxetine, fluvoxamine, tryptophan, 6-nitroquipazine, 5-hydroxy tryptophan, citalopram, and clomipramine are useful for administration to alleviate symptoms of MCI.

In one aspect of the invention, the symptoms of MCI can be treated with any 5-HT₁ agonist or partial agonist, including, but not limited to: 8-hydroxy-2-(di-n-propylamino) tetralin; sumatriptan; 2-[5-[3-(4-methylsulfonylamino) benzyl-1,2,4-oxadiazol-5-yl]-1H-indol-3yl] ethanamine; (s)-3, 4-dihydro-1-[2-[4-(4-methoxyphenyl)-1-piperazinyl] ethyl]-N-methyl-1H-2benzopyran-6-carboximide; 5-(4-flurobenzoyl) amino-3-(1-methylpiperidin-4-yl)-1H-indole fumarate; Anpirtoline; BMY 7378; BP-554; 3-(1-methylpiperidin-4-yl)1H-indol-5-ol; Buspirone; 5-Carboxamidotryptamine; CGS-12066B; 1-(3-Chlorophenyl)-4-hexylpiperazine; CP 93129; GR 46611; (R)-(+)-8-hydroxy-2-(di-n-propylamino) tetralin; 8-hydroxy-PIPAT; MDL 73005EF (±)-5-Methoxy-3-dipropylaminochroman; (±)-8-Methoxy-2-dipropylaminotetralin; 5-Nonyloxytryptamine; RU 24969; TFMPP; or combinations thereof.

In one aspect of the invention, the symptoms of MCI can be treated with any 5-HT₂, 5-HT₃, or 5-HT₄ agonist, or partial agonist thereof, including, but not limited to: (s)-2-(6-chloro-5-fluroindol-1-yl)-1-methylethylamine; 4-amino-(6-chloro-2-pyridyl)-1-piperidine hydrochloride; (endo-N-8-methyl-8-azabicyclo[3.2.1]oct-3-yl)-2,3-dihydro-3-isopropyl-2-oxo-1H-benzimidazol-1-carboxamide hydrochloride; 2-(1-piperidinyl)ethyl-4-amino-5-chloro-2-methoxybenzoate; 1-[5(2-thienylmethoxy)-1H-3-indolyl]propan-2-amine hydrochloride; mCPP; β-Methyl-5-hydroxytryptamine; MK 212; m-Chlorophenylbiguanide; 2-Methyl-5-hydroxytryptamine; N-Methylquipazine; Phenylbiguanide; Quipazine; RS 56812; 2[1-(4-Piperonyl)piperazinyl]benzothiazole; RS 67333; 1-(4-amino-5-chloro-2-methoxyphenyl)-3-(1-nbutyl-4-piperidinyl)-1-propanone; or combinations thereof.

In one aspect of the invention, the symptoms of MCI can be treated with: Bufotenine monooxalate; 1-(m-Chlorophenyl)-bigunide HCl; 1-(3-Chlorophenyl)-piperazine HCl; CGS-12066B maleate; N,N-Dipropyl-5-carboxamidotryptamine maleate; DOI HCl, (±)-; DOI HCl, R(−)-; DOI HCl, S(+)-; DOB HBr, (±)-; DMA HCl; 5-HTQ iodide; 8-hydroxy-2-(di-npropylamino) tetralin HBr, (±)-; 8-hydroxy-2-(di-n-propylamino) tetralin HBr, S(−)-; D-Lysergic acid diethylamide tartrate; Mescaline sulfate; 5-Methoxy DMT; 1-(2-Methoxyphenyl)-piperazine HCl; 5-Methoxytryptamine HCl; 2-Methylserotonin maleate; α-Methylserotonin maleate; 5-(Nonyloxy)-tryptamine hydrogen oxalate; Oxymetazoline HCl; PAPP (LY-165, 163); 1-Phenylbiguanide; Quipazine dimaleate; Quipazine, N-methyl dimaleate; SC 53116; Serotonin creatinine sulfate; Serotonin HCl; Serotonin oxalate; Spiroxatrine; UH-301 HCl, R(+)-; Urapidil HC]; Urapidil, 5-methyl-; WB-4101 HCl; or combinations thereof.

One skilled in the art will recognize that various salts of the above serotonergic agents can be used. Moreover, stereoisomers of the above salts are suitable in the invention.

The serotonergic agents of the invention can be administered in any suitable form including oral and parenteral.

In one aspect of the invention, the effective doses of the serotonergic agents of the invention range from about 10 micrograms per kilogram body weight to about 100 milligrams per kilogram body weight. In one aspect of the invention the effective doses range from about 0.1 milligram per kilogram of body weight to about 10 milligrams per kilogram of body weight. The effective dose can be from about 0.5 milligrams per kilogram of body weight to about 4 milligrams per kilogram of body weight. In one embodiment the effective dose can be from about 10 micrograms per kilogram of body weight to about 500 micrograms per kilogram of body weight. In one embodiment the effective dose can be from about 5 milligrams per kilogram of body weight to about 100 milligrams per kilogram of body weight.

8.11. Water Maze for Evaluation of Memory

Experimental Procedure

Spatial memory is assessed in a water maze, commonly termed a Morris water maze, or variations thereof. The nootropic effect of the natural products of the invention is assessed in the water maze. The memory of a rat is evaluated by testing its ability to find a submerged escape platform in a water tank using prior training and spatial clues from outside the maze. Success in the water maze test depends on memory of the location of the submerged escape platforms, and also on motor skills and exploration abilities.

The water maze is a round pool about 1.5 m in diameter with various large spatial cues on the walls of the room. The hidden escape platform is made of clear plastic with a friction surface and is submerged just under the water level. The water temperature is maintained at about 78° F. The room is uniformly and dimly lit.

Training for Search Behavior

To begin the training, the rat is placed to swim in the maze for up to one minute without the visual clues on the walls. After finding and climbing the platform, the rat is returned to its cage. The rats are trained three times per day for four days. The location of the submerged platform and the point of entry of the animal into the maze are changed for each training session. Thus, in the course of Morris water maze training, animals develop a search behavior. Animals with similar levels of search skills are used for further studies.

One of the two groups is then randomly allocated to experimental (e.g. dexnorfenfluramine), while the other is used as a control group.

Spatial Training

The trained animals are randomly assigned to an experimental or a control group. The spatial training is begun at three days after the training period. The tests continue for eight days with three trials each day. During the test period, the platform is placed in the same position in the pool, the rats are released at randomly chosen points, and the rats are allowed to swim for one minute.

The day after the end of spatial training, all rats are given a probe trial to assess their development of spatial bias. During the probe trial, the platform is removed from the pool and each rat is given 60 seconds of swimming.

Test for Memory Retention

After completion of the learning acquisition phase, all animals are kept for 35 days in their home cages with food and water ad libitum, but without experimental drugs. After that period, all animals are given a four-day retention test with three tests each day, in the same experimental room, with all spatial cues and platform position in the same location as in the behavior learning training. The handling procedure before and during the retention test is similar to the training procedures, but no drugs are given to the rats.

Data Measurement and Analysis

Swimming behavior is videotaped for analysis and archiving. Measurements are made of latency (time to search for the platform), the time spent in each sector of the pool, the mean swim path, swimming speed, float time (that is, at forward progress of less than 5 cm/s), and platform proximity (that is, change in spatial bias). Measurements are made by the methods standard in the art. The accuracy of the search is evaluated by a sector preference time and a site preference scalar, which counts instances of proximity to the presumptive platform. Data are analyzed by analysis of variance.

Experimental Observation

The effects of dexnorfenfluramine on rat search behavior are addressed by analysis of rat search behavior and swimming ability after administration of dexnorfenfluramine. In a first test, four rats (about 24 months old) are used. The animals are handled before the experiment, and undergo non-spatial training for four days with three trials per day. Dexnorfenfluramine is injected at 2 mg/kg body weight. The rats are trained for four days with three trials per day. Statistical comparisons are supplemented with anecdotal observations. After the last training trial, the rats are given a probe trial.

Thirty days after the probe trial, the rats are given a retention test, as above, in the absence and presence of dexnorfenfluramine.

Induced Memory Impairment

Rats are injected with ibotenic acid into the medial septal area according to a standard model for impaired memory. Training in the maze is performed for three days, two weeks after treatment with ibotenic acid. The memory sparing effect of injection with dexnorfenfluramine is evaluated using the methods indicated above.

9.0 Conclusion

Accordingly, the invention provides compositions and methods for preventing, delaying, alleviating, or inhibiting abnormal APP synthesis by the administration of serotonergic agents that stimulate formation or secretion of APPs and prevents, delays, alleviates, or ameliorates non-disease or pre-disease conditions including, but not limited to MCI and occlusive stroke.

It should be apparent that other embodiments of the invention can be readily contemplated by those of ordinary skill in the art after reviewing the present specification and teachings. The present invention is not limited, however, to the specific embodiments presented herein and should not be construed so narrowly as to exclude embodiments that fall within the scope and spirit of the invention, which invention is limited solely by the following claims.

We claim:

1. A method of preventing, delaying, attenuating or ameliorating the symptoms of Mild Cognitive Impairment in a subject comprising:
administering to the subject an effective amount of an agent that stimulates soluble amyloid precursor protein secretion,
whereby the impairment is prevented, attenuated or ameliorated.

2. The method of claim 1 in which the agent is a serotonergic agonist, serotonergic partial agonist, serotonin reuptake inhibitor, or combination thereof.

3. The method of claim 2 in which the agent is dexnorfenfluramine; dexfenfluramine; fenfluramine; fluoxetin; sertraline; paroxetine; fluvoxamine; tryptophan; 6-nitroquipazine; 5-hydroxy tryptophan; citalopram; clomipramine; 8-hydroxy-2-(di-n-propylamino) tetralin; sumatriptan; 2-[5-[3-(4-methylsulfonylamino) benzyl-1,2,4-oxadiazol-5-yl]-1H-indol-3yl] ethanamine; (s)-3, 4-dihydro-1-[2-[4-(4-methoxyphenyl)-1-piperazinyl]ethyl]-N-methyl-1H-2-benzopyran-6-carboximide; 5-(4-flurobenzoyl) amino-3-(1-methylpiperidin-4-yl)-1H-indole fumarate; Anpirtoline; BMY 7378; BP-554; 3-(1-methylpiperidin-4-yl)1H-indol-5-ol; Buspirone; 5-Carboxamidotryptamine; CGS-12066B; 1-(3-Chlorophenyl)-4-hexylpiperazine; CP 93129; GR 46611; (R)-(+)-8-hydroxy-2-(di-n-propylamino) tetralin; 8-hydroxy-PIPAT; MDL 73005EF; (±)-5-Methoxy-3-dipropylaminochroman; (±)-8-Methoxy-2-dipropylaminotetralin; 5-Nonyloxytryptamine; RU 24969; TFMPP; (s)-2-(6-chloro-5-fluroindol-1-yl)-1-methylethylamine; 4-amino-(6-chloro-2-pyridyl)-1-piperidine hydrochloride; (endo-N-8-methyl8-azabicyclo[3.2.1]oct-3-yl)-2,3-dihydro-3-isopropyl-2-oxo-1H-benzimidazol-1-carboxamide hydrochloride; 2-(1-piperidinyl)ethyl-4-amino-5-chloro-2-methoxybenzoate; 1-[5(2-thienylmethoxy)-1H-3-indolyl]propan-2-amine hydrochloride; mCPP; α-Methyl-5-hydroxytryptamine; MK 212; m-Chlorophenylbiguanide; 2-Methyl-5-hydroxytryptamine; N-Methylquipazine; Phenylbiguanide; Quipazine; RS 56812; 2[1-(4Piperonyl)piperazinyl]benzothiazole; RS 67333; 1-(4-amino-5-chloro-2-methoxyphenyl)-3-(1-nbutyl-4-piperidinyl)-1-propanone; Bufotenine monooxalate; 1-(m-Chlorophenyl)-bigunide HCl; 1-(3-Chlorophenyl)-piperazine HCl; CGS-12066B maleate; N,N-Dipropyl-5-carboxamidotryptamine maleate; DOI HCl, (±)-; DOI HCl, R(−)-; DOI HCl, S(+)-; DOB HBr, (+)-; DMA HCl; 5-HTQ iodide; 8-hydroxy-2-(di-n-propylamino) tetralin HBr, (±)-; 8-hydroxy-2-(di-n-propylamino) tetralin HBr, S(−)-; D-Lysergic acid diethylamide tartrate; Mescaline sulfate; 5-Methoxy DMT oxalate; 1-(2-Methoxyphenyl)-piperazine HCl; 5-Methoxytryptamine HCl; 2-Methylserotonin maleate; α-Methylserotonin maleate; 5-(Nonyloxy)-tryptamine hydrogen oxalate; Oxymetazoline HCl; PAPP (LY-165, 163); 1-Phenylbiguanide; Quipazine dimaleate; Quipazine, N-methyl dimaleate; SC 53116; Serotonin creatinine sulfate; Serotonin HCl; Serotonin oxalate; Spiroxatrine; UH-301 HCl, R(+)-; Urapidil HCl; Urapidil, 5-methyl-; WB-4101 HCl; or combinations thereof.

4. The method of claim 1 in which the effective amount is at least about one microgram per kilogram of body weight.

5. The method of claim 1 in which the effective amount is up to about 100 milligrams per kilogram of body weight.

6. The method of claim 1 in which the effective amount is at least about 100 micrograms per kilogram of body weight.

7. The method of claim 1 in which the administration is oral, enteral, parenteral, topical, or combinations thereof.

8. A method of increasing the level of soluble amyloid precursor protein in the cerebrospinal fluid of a subject comprising:
administering to the subject an effective amount of an agent that stimulates secretion of soluble amyloid precursor protein into the cerebrospinal fluid, whereby the level of soluble amyloid precursor protein in the cerebrospinal fluid is increased.

9. The method of claim 8 in which the agent is a serotonergic agonist, partial agonist, serotonin reuptake inhibitor, or combinations thereof.

10. The method of claim 8 in which the agent is not a non-steroidal anti-inflammatory agent.

11. The method of claim 9 in which the agent is dexnorfenfluramine; dexfenfluramine; fenfluramine; fluoxetin; sertraline; paroxetine; fluvoxamine; tryptophan; 6-nitroquipazine; 5-hydroxy tryptophan; citalopram; clomipramine; 8-hydroxy-2-(di-n-propylamino) tetralin; sumatriptan; 2-[5-[3-(4-methylsulfonylamino) benzyl-1,2,4-oxadiazol-5-yl]-1H-indol-3yl] ethanamine; (s)-3, 4-dihydro-1-[2-[4-(4-methoxyphenyl)-1-piperazinyl]ethyl]-N-methyl-1H-2-benzopyran-6-carboximide; 5-(4-flurobenzoyl) amino-3-(1-methylpiperidin-4-yl)-1H-indole fumarate; Anpirtoline; BMY 7378; BP-554; 3-(1-methylpiperidin-4-yl)1H-indol-5-ol; Buspirone; 5-Carboxamidotryptamine; CGS-12066B; 1-(3-Chlorophenyl)-4-hexylpiperazine; CP 93129; GR 46611; (R)-(+)-8-hydroxy-2-(di-n-propylamino) tetralin; 8-hydroxy-PIPAT; MDL 73005EF; (±)-5-Methoxy-3-dipropylaminochroman; (±)-8-Methoxy-2-dipropylaminotetralin; 5-Nonyloxytryptamine; RU 24969; TFMPP; (s)-2-(6-chloro-5-fluroindol-1-yl)-1-methylethylamine; 4-amino-(6-chloro-2-pyridyl)-1-piperidine hydrochloride; (endo-N-8-methyl-8-azabicyclo[3.2.1]oct-3-yl)-2,3-dihydro-3-isopropyl-2-oxo-1H-benzimidazol-1 -carboxamide hydrochloride; 2-(1-piperidinyl)ethyl-4-amino-5-chloro-2-methoxybenzoate; 1-[5(2-thienylmethoxy)-1H-3-indolyl]propan-2-amine hydrochloride; mCPP; α-Methyl-5-hydroxytryptamine; MK 212; m-Chlorophenylbiguanide; 2-Methyl-5-hydroxytryptamine; N-Methylquipazine; Phenylbiguanide; Quipazine; RS 56812; 2[1-(4-Piperonyl)piperazinyl]benzothiazole; RS 67333; 1-(4-amino-5-chloro-2-methoxyphenyl)-3 -(1-nbutyl-4-piperidinyl)-1-propanone; Bufotenine monooxalate; 1-(m-Chlorophenyl)-bigunide HCl; 1-(3-Chlorophenyl)-piperazine HCl; CGS-12066B maleate; N,N-Dipropyl-5-carboxamidotryptamine maleate; DOI HCl, (±)-; DOI HCl, R(−)-; DOI HCl, S(+)-; DOB HBr, (±)-; DMA HCl; 5-HTQ iodide; 8-hydroxy-2-(di-n-propylamino) tetralin HBr, (±)-; 8-hydroxy-2-(di-n-propylamino) tetralin HBr, S(±)-; D-Lysergic acid diethylamide tartrate; Mescaline sulfate; 5-Methoxy DMT oxalate; 1-(2-Methoxyphenyl)-piperazine HCl; 5-Methoxytryptamine HCl; 2-Methylserotonin maleate; α-Methylserotonin maleate; 5-(Nonyloxy)-tryptamine hydrogen oxalate; Oxymetazoline HCl; PAPP (LY-165, 163); 1-Phenylbiguanide; Quipazine dimaleate; Quipazine, N-methyl dimaleate; SC 53116; Serotonin creatinine sulfate; Serotonin HCl; Serotonin oxalate; Spiroxatrine; UH-301 HCI, R(+)-; Urapidil HCl; Urapidil, 5-methyl-; WB-4101 HCl; or combinations thereof.

12. The method of claim 8 in which the effective amount is at least about one microgram per kilogram of body weight.

13. The method of claim 8 in which the effective amount is up to about 100 milligrams per kilogram of body weight.

14. The method of claim 8 in which the effective amount is at least about 100 micrograms per kilogram of body weight.

15. A method of decreasing the level of soluble amyloid precursor protein in the cerebrospinal fluid comprising: administering an effective amount of a serotonin antagonist.

16. The method of claim 15 in which the effective amount is between about one microgram per kilogram body weight to about 10 milligrams per kilogram body weight.
